(19) United States
(12) Patent Application Publication
(10) Pub. No.: US 2008/0300569 A1
(43) Pub. Date: Dec. 4, 2008

(54) Storage-Stable Oral Dosage Form of Amoxicillin and Clavulanic Acid

(75) Inventors: Dieter SCHATEIKIS, Stolberg (DE); Elke Sternberger, Wuerselen (DE); Iris Ziegler, Neusaess (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 12/192,784

(22) Filed: Aug. 15, 2008

Related U.S. Application Data
(63) Continuation of application No. PCT/EP2007/001335, filed on Feb. 15, 2007.

(30) Foreign Application Priority Data
Feb. 17, 2006 (DE) 10 2006 007 830.6

(51) Int. Cl. A61B 19/00

(52) U.S. Cl. 604/403

(57) ABSTRACT

An airtight-packaged system including:
(1) a combination of two independently prepared pharmaceutical drug forms composed of a first drug form in aqueous formulation containing amoxicillin trihydrate as active pharmaceutical ingredient and a second drug form prepared separately from the first and containing potassium clavulanate as active pharmaceutical ingredient, in which the active ingredients in the combination of the first and second drug forms are present in a nominal weight ratio of amoxicillin to clavulanic acid of from 20:1 to 1:1, and
(2) a preparation containing at least one desiccant in amounts such that when the packaged system is stored under stress conditions at 40° C. and 75% relative humidity for up to 3 months, the dimerization of amoxicillin trihydrate does not exceed 1.5% by weight, and the degradation of clavulanic acid does not exceed 10% by weight.

### CROSS REFERENCE TO RELATED APPLICATIONS

This application is a continuation of international patent application no. PCT/EP2007/001335, filed Feb. 15, 2007, designating the United States of America, and published in German on Aug. 23, 2007 as WO 2007/093425, the entire disclosure of which is incorporated herein by reference. Priority is claimed based on Federal Republic of Germany patent application no. DE 10 2006 007 830.6, filed Feb. 17, 2006.

### FIELD OF THE INVENTION

The present invention relates to an airtight packaged system comprising:
1. a combination of two mutually independently prepared, multiparticulate, pharmaceutical dosage forms consisting of an aqueously formulated dosage form containing amoxicillin trihydrate as pharmaceutical active ingredient and of a dosage form prepared, preferably formulated, separately therefrom and containing potassium clavulanate as pharmaceutical active ingredient, the active ingredients being present in the combination in a nominal weight ratio of amoxicillin to clavulanic acid of from 20:1 to 1:1, and
2. a preparation comprising at least one desiccant in such quantities that, on storage of the packaged system under stress conditions of 40° C./75% relative atmospheric humidity for up to 3 months, amoxicillin trihydrate is dimerised at most up to 1.5 wt. % and the clavulanic acid is degraded to at most 10 wt. %.

### BACKGROUND OF THE INVENTION

Formulations comprising the antibiotic amoxicillin, as amoxicillin trihydrate, and potassium clavulanate as β-lactamase inhibitor, are known, oral medicines for treating bacterial infections, such as otitis media, sinusitis and infections of the upper and lower airways, of the urinary system or of the skin both in children and in adults.

Depending on the dosage and/or the severity of the complaint, such medicines are administered two or three times daily. The pharmaceutical composition comprising amoxicillin trihydrate and potassium clavulanate with conventional auxiliary substances as a powder mixture may be press-molded into tablets or simply made available as a dry powder mixture, which is dispersed in water or aqueous liquids before it is first taken. This dispersion is suitable for continuous treatment above all of children and patients with swallowing difficulties, since such patient groups find it very difficult to take tablets.

Marketed pharmaceutical formulations include formulations which contain the two active ingredients, amoxicillin trihydrate and potassium clavulanate, in a nominal ratio by weight of 4:1, 6:1, 7:1 and 8:1, expressed as amoxicillin and clavulanic acid. As a daily dosage, amoxicillin may be taken in quantities of 15 to 80 mg/kg/day with a corresponding pro rata quantity of clavulanic acid.

The corresponding pharmaceutical formulations, comprising the two active ingredients as dry powder active ingredient mixtures, are preferably present in air tight containers, to which a desiccant may be added in order to rule out decomposition of the potassium clavulanate due to water vapor in the atmosphere. It is known that potassium clavulanate in particular is highly water-sensitive and decomposes on exposure to moisture, such as for example water vapor. Accordingly, potassium clavulanate must be produced and formulated with amoxicillin trihydrate under the lowest possible atmospheric humidity and the lowest possible temperatures, preferably below 20° C. and a relative atmospheric humidity of at most 20%. For this reason, the potassium clavulanate itself is also prediluted with colloidal silicon dioxide or microcrystalline cellulose as desiccant, in order largely to prevent decomposition of the potassium clavulanate due to atmospheric water vapor during storage thereof and during further formulation with amoxicillin trihydrate.

As already explained, the dry powder active ingredient mixtures are preferably distributed in airtight containers, from which the particular dose is taken after dispersion in an aqueous liquid. Dosage inaccuracies may thus occur during subsequent treatment.

However, even if such dry powder active ingredient mixtures are made available packaged as individual doses in airtight sachets, so minimising the risk of decomposition of potassium clavulanate through the influence of atmospheric water vapor or aqueous dispersion medium, such administration forms also display serious disadvantages when being taken. Since even such an individual dose or single dose of the dry powder mixture must be swallowed with the assistance of an aqueous liquid, it cannot always be ensured that the entire single dose necessary for treatment has been taken by the patient. It is also impossible to avoid the perception of a typical penicillin taste of the antibiotic despite aromatisation of these powder mixtures. By administering tablets, more precise treatment could be achieved, but is barely possible, given in particular the swallowing behavior of children and patients with swallowing difficulties, since for such patient the particles to be swallowed must not be too large overall. Given the high active ingredient dose required each time amoxillin is taken, this can only be achieved by the highest possible active ingredient loading of the individual particles of a multiparticulate dosage form, for example.

Only in this way is it possible to ensure that the number of particles in a single dose is not too high, but above all that the volume and mass of the total dose is kept as small as possible. When using a clavulanic acid component diluted for the purpose of stabilization to produce a pharmaceutical formulation comprising potassium clavulanate and amoxicillin trihydrate it is barely possible, however, to make such multiparticulate dosage forms with in each case an elevated active ingredient loading available as a single dose. To this end, recourse has to be made to the above-described powder mixtures as the basis for the dispersion.

### SUMMARY OF THE INVENTION

The object of the present invention was accordingly to provide, preferably as a single dose, storage-stable, preferably multiparticulate administration forms comprising amoxicillin trihydrate and potassium clavulanate, which are not powder mixtures of the active ingredients, which do not display the stated disadvantages and can therefore readily be taken by children and by patients with swallowing difficulties.

This and other objects are achieved in accordance with the present invention by providing the airtight packaged system as described and claimed hereinafter. According to the invention, the airtight packaged system comprises:
1. a combination of two mutually independently prepared, multiparticulate, pharmaceutical dosage forms consisting of an aqueously formulated dosage form containing amoxicillin trihydrate as pharmaceutical active ingredient and of a dosage form prepared, preferably formulated, separately therefrom and containing potassium clavulanate as pharmaceutical active ingredient, the active ingredients being present in the combination in a nominal weight ratio of amoxicillin to clavulanic acid of from 20:1 to 1:1, and
2. a preparation comprising at least one desiccant in such quantities that, on storage of the packaged system under stress conditions of 40° C./75% relative atmospheric humidity for up to 3 months, amoxicillin trihydrate is dimerised at most up to 1.5 wt. % and the clavulanic acid is degraded to at most 10 wt. %.

The system according to the invention is distinguished in that the two active ingredients, amoxicillin trihydrate and potassium clavulanate, are not present as a powder mixture but rather are in each case separately prepared, preferably formulated in such a way to yield a multiparticulate administration form that the desired elevated active ingredient loading of the individual particles is achieved with the respective active ingredient and the mass or the volume of the individual particles is not increased to such an extent as to result in the above-mentioned patient groups in particular taking an incomplete dose. This is achieved inter alia in that, for each of the two active ingredients, the formulation variant most favorable to high active ingredient loading may be selected, regardless of the second active ingredient of the combination, the two preparations, preferably formulations, then only subsequently being brought together in the desired ratio. This means in the present case that the amoxicillin trihydrate may be formulated aqueously, such as for example by means of aqueous granulation, regardless of the extreme water sensitivity of the potassium clavulanate. On the other hand, the potassium clavulanate may preferably be undiluted, i.e. used as crystals or formulated with elevated active ingredient loading, provided that this takes place at temperatures of below 25° C., preferably below 20° C., and a low relative atmospheric humidity, without any traces of water, which lead to decomposition of the potassium clavulanate, being introduced by the 2nd active ingredient, amoxicillin. Consequently, effective protection of the administration form comprising the two dosage forms against the effects of atmospheric water vapor is necessary only once the two dosage forms have been brought together to yield a combined dose and during subsequently storage.

This effective protection for potassium clavulanate and also for amoxicillin trihydrate upon storage of the system according to the invention, even when packaged in airtight manner, is achieved according to the invention in that a desiccant preparation is added to the system as a further system component in such quantities that, upon storage of the airtight packaged system under stress conditions at 40° C./75% relative atmospheric humidity for up to 3 months, amoxicillin trihydrate is dimerized at most up to 1.5 wt. %, preferably up to 1.0 wt. %, particularly preferably up to 0.5 wt. %, once the protective water of hydration has been wholly or partially removed, and the clavulanic acid is degraded to at most 10 wt. %, preferably to 5 wt. %.

This is achieved by the use of a desiccant preparation, which comprises a desiccant which absorbs at least 25 mg water per gram of desiccant, preferably at least 27 mg water per gram of desiccant, both at 40° C./75% relative atmospheric humidity and at 22° C./80% relative atmospheric humidity in each case within the first 24 hours of storage of an airtight packaged system according to the invention under the stated conditions and at least 40 mg water per gram of desiccant, preferably at least 50 mg water per gram of desiccant during subsequent storage of up to a total of 7 days in each case under the stated conditions, the water absorption of the desiccant at most doubling within the first 24 hours or after a total of 7 days in each case on changeover of the storage conditions from 22° C./80% relative atmospheric humidity to 40° C./75% relative atmospheric humidity.

The use of such a desiccant ensures not only that at least the content of unbound water, which is present in the airtight packaged system according to the invention in particular as a consequence of the aqueously formulated amoxicillin trihydrate dosage form, is absorbed without decomposition of the potassium clavulanate being initiated by traces of water but also that the amoxicillin trihydrate is not dehydrated, which results in the above-mentioned dimeric decomposition products. Such degradation may namely be observed when conventional desiccant is used, such as for example molecular sieves, above all under stress storage conditions, which are the storage conditions required under the ICH1 Guidelines.

Preferably, a suitable desiccant is an amorphous silica, preferably a precipitated silica or a silica gel or a pyrogenic silica. Very particularly preferably, silica gel is suitable as the amorphous silica. Amorphous silica products are commercially obtainable and are distributed inter alia under the tradenames “Syloid™” (by Grace Davidson, USA) or “Aerosil™” (by Degussa AG, Germany).

In order to achieve the maximum possible active ingredient loading of the respective multiparticulate dosage form of the clavulanic acid component or of amoxicillin trihydrate, the respective active ingredients are preferably formulated to yield granules, which are optionally subjected to further processing to yield the administration form used. These administration forms may also be pellets or microtablets produced from the respective granules by press-molding.

Such granules, in particular if they are round granules, offer the advantage of a smaller surface area, better flowability and easier swallowing than powder mixtures, which also leads to reduced taste perception. This allows taking without the addition of aromatizing aids. Since taking the clavulanic acid component does not have a negative effect on taste, this active ingredient may also be used as crystals, i.e. as active ingredient crystals.

When the potassium clavulanate, which is used undiluted to achieve an elevated dosage, is granulated, there is no question of wet granulation due to the water sensitivity of the potassium clavulanate. Accordingly, the granules containing potassium clavulanate may be produced by melt granulation as far as possible with exclusion of atmospheric water vapor at a relative atmospheric humidity of ≦30%, preferably of ≦20%. The auxiliary materials used should as far as possible be anhydrous auxiliary materials, which are preferably optionally redried immediately before use and, in the case of the binder, melt at temperatures of below 65° C. Sucrose fatty acid esters are very particularly suitable for this purpose. These sucrose fatty acid esters may be mono-, di-, tri- or polyesters of sucrose with at least one fatty acid, preferably a saturated or unsaturated fatty acid with C₁₂-C₂₂, preferably a saturated C₁₂-C₂₂ fatty acid. Preferably, a mixture of at least 2 of the stated mono-, di-, tri- or polyesters of sucrose is used. Particularly preferably, sucrose fatty acid esters of stearic acid and/or palmitic acid are used.

Sucrose fatty acid esters with an HLB value of less than or equal to 3, preferably less than or equal to 2, very particularly preferably of roughly 1 are particularly suitable. In addition to sucrose fatty acid esters, further physiological auxiliary materials selected from the group comprising lactose, microcrystalline cellulose, silicon dioxide, CaHPO₄, kaolin, talcum, titanium dioxide, mannitol, pH regulators, preferably citric acid, Na₂HPO₄ or ascorbic acid, preferably kaolin and/or CaHPO₄ may optionally also be used.

Furthermore, to produce the granules, fillers may also be used, which additionally make granulation easier.

To produce the granules, the binder is optionally melted with the optionally present other auxiliary substances and preferably granulated in a closed system with the potassium clavulanate in a suitable granulator, preferably at elevated rotational or chopper speeds. The elevated chopper speeds are desirable in order to obtain particles which are as far as possible uniform and as far as possible have a particle size of below 1000μ. The resulting granules may optionally be rounded to yield pellets, before they are cooled and classified using the screening method.

Preferably, the combined 250 to 800 μm screening fractions are put to further use.

A preferred composition of potassium clavulanate pellets and a method for the production thereof is described in US patent publication no. US 2006/0147524 (=DE 103 41 264), the disclosure of which is incorporated herein by reference.

Sufficient bioavailability is ensured as a result of the rapid disintegration of the pellets in the gastrointestinal tract.

As has already been mentioned, the potassium clavulanate is used undiluted, i.e. without addition of desiccant.

When producing the oral dosage form of amoxicillin, as amoxicillin trihydrate, aqueously formulated separately from potassium clavulanate and preferably multiparticulate, it must be ensured, in the case of the desired, high-dosage dosage forms, preferably in the form of granules or microtablets produced therefrom by press-molding, particularly preferably in the form of pellets, preferably in a smooth, spherical form, that such dosage forms guarantee sufficient bioavailability. This is achieved in particular in that the active ingredient-containing particles disintegrate rapidly, preferably within 30 minutes, at a pH value in the upper small intestine, in which absorption of the active ingredient predominantly takes place, and release the active ingredient. Of known formulation aids, which are used in particular in the production of granules or pellets, such as microcrystalline cellulose, low-substituted hydroxypropylcellulose, hydroxypropylmethylcellulose, it is familiar to a person skilled in the art that these extrusion or spheronizing agents do indeed result in granules or pellets with the desired shape, namely spherical and with a smooth surface, but that, when using these auxiliary materials it is not always ensured that multiparticulate dosage forms disintegrate rapidly enough in the upper region of the small intestine for sufficient bioavailability to be achieved.

However, if, when producing the multiparticulate dosage form, preferably granules or pellets, very particularly preferably extruded pellets, carrageenan, preferably kappa-carrageenan, is also used, particularly preferably in a quantity of from 5 to 30 wt. % relative to the total weight of the dosage form, not only is granulation, optionally with subsequent extrusion and spheronization, possible, but also the necessary rapid disintegration of the multiparticulate dosage form is achieved at pH values which correspond to those in the upper region of the small intestine despite an elevated active ingredient loading.

Rapid disintegration of such dosage forms and thus release of the active ingredient amoxicillin is further promoted in that, in addition to carrageenan, the multiparticulate dosage form also contains tricalcium phosphate. Preferably, the weight ratio of tricalcium phosphate to carrageenan in the multiparticulate dosage forms should amount to 1:1 to 1:10, particularly preferably 1:2 to 1:6. This enables complete release of the active ingredient within 15 minutes.

In addition to the combination of carrageenan and optionally tricalcium phosphate, the multiparticulate dosage form of amoxicillin trihydrate may further comprise fillers, binders, slip agents, dyes and/or preservatives as auxiliary substances.

Because of an undesired influence on the rate of disintegration and thus on the release of amoxicillin trihydrate, the multiparticulate dosage forms are preferably produced without using microcrystalline cellulose or other spheronization auxiliaries, such as low-substituted hydroxypropylcellulose, hydroxypropyl-methylcellulose, hydroxypropylcellulose, powdered cellulose, sodium carboxy-methylcellulose and/or polyvinylpyrrolidone. These auxiliary materials may, however, be present in a coating, for example in a coating for neutralizing taste and/or for providing corresponding multiparticulate dosage forms of amoxicillin trihydrate with a finish which is resistant to gastric juices.

In a preferred embodiment, the multiparticulate dosage forms, preferably the granules or pellets of amoxicillin trihydrate, are provided with at least one coating, particularly preferably with a coating which is taste-neutralized and/or resistant to gastric juices. The coatings may be applied in a quantity of from 1 to 50 wt. %, relative to the total weight of the dosage form, depending on the type and function of the coating, for providing a taste-neutralized finish preferably in a quantity of from 1 to 5 wt. %, preferably 1 to 3 wt. %, relative to the total weight of the dosage form.

Suitable materials for a gastric juice-resistant or taste-masking coating are preferably methacrylic acid/alkyl (meth)acrylate copolymers, particularly preferably copolymers of methacrylic acid/methyl methacrylate with a ratio of 1:1 to 1:2 such as Eudragit L® or Eudragit S®, very particularly preferably copolymers of methacrylic acid/ethyl acrylate 1:1 such as Eudragit L55®, Eudragit L30D-55®, which dissolve rapidly at a pH value of ≧5.5. Coatings based on celluloses or based on shellac, which are known to a person skilled in the art, may furthermore be applied as gastric juice-resistant coatings. The coatings may be applied with appropriate solutions or dispersions of the polymers in an organic or aqueous medium, an aqueous medium being preferred. Suitable saliva-resistant coatings are preferably coatings based on Eudragit E or Eudragit EP0.

Persons skilled in the art are aware that conventional plasticizers, dyes, slip agents, such as talcum, magnesium stearate and/or glycerol monostearate should or may be added to known coating materials.

According to the invention, “gastric juice” is taken to mean both the natural composition of gastric juice and the artificial preparations similar to gastric juice (pH 1.2-2) which are familiar to a person skilled in the art. Likewise, “release in the small intestine” is taken to mean both release in natural small intestine juice and release in preparations similar to small intestine juice at pH values of 6-7, preferably pH 6.4-6.8, as are defined in relevant pharmacopoeias.

The dosage forms produced additionally using carrageenan and optionally tricalcium phosphate are distinguished in that they have an elevated rate of disintegration, whereby at least 85% of the antibiotic, amoxicillin, is released within 30 minutes, once any optionally present coating has previously dissolved. This elevated disintegration rate preferably occurs at pH values of 6.4 to 6.8.

The dosage forms containing amoxicillin trihydrate are produced by mixing and granulating the starting materials. Granulation is preferably performed as wet granulation, with water or aqueous solvents being used as granulating liquids. Suitable solvents are known to a person skilled in the art. The advantage of wet granulation using water or an aqueous solvent is inter alia that no organic solvent or only readily removable organic solvents are used in production of the administration forms.

After granulation, the granular product may optionally also be extruded, the extrudates isolated and optionally shaped, preferably spheronized or the dried granular product press-molded into microtablets. If necessary, the multiparticulate dosage form may also be classified, preferably using the screening method, the particles of the 250 μm to 710 μm screening fractions being used according to the invention as the dosage form.

The multiparticulate amoxicillin dosage form may preferably also be protected with a coating, above all to provide optimum taste masking for when the dosage form is taken with beverages other than water, such as for example soft drinks or fruit juices.

It is known to persons skilled in the art that the components of the amoxicillin trihydrate-containing dosage form may be mixed simultaneously or in succession. Production of the mixture may proceed in known mixers or granulators, such that optionally mixing, granulation and optionally extrusion may proceed simultaneously.

The optionally performed extrusion and/or spheronization of the granules and the coating thereof with gastric juice-resistant and/or taste-concealing coatings may in each case be performed in the apparatus known to a person skilled in the art. A fluidized bed apparatus may preferably be used for applying a coating. Preferred amoxicillin trihydrate-containing dosage forms, preferably granules or pellets, and methods for the production thereof are disclosed in German patent application 10 2005 042 875. The corresponding disclosure is hereby introduced as a reference and is deemed to be part of the present disclosure.

To produce the storage-stable system according to the invention, the two mutually independently produced, separately formulated dosage forms, which are multiparticulate but not present as active ingredient powders and which in each case contain amoxicillin trihydrate or potassium clavulanate, are preferably brought together only directly prior to airtight packaging of the system in a nominal weight ratio of amoxicillin to clavulanic acid of from 20:1 to 1:1, preferably in a nominal weight ratio of 4:1, 6:1, 7:1 or 8:1, and, prior to airtight packaging, provided with a preparation comprising at least one desiccant of the above-described type. The in each case multiparticulate, formulated, particularly preferably granulated, active ingredient-containing dosage forms may be brought together in single dose form in a sachet, wherein the desiccant preparation may be added likewise in multiparticulate form to the two active ingredient-containing dosage forms as a finish for the sachets, for example as desiccant strips, or as a physiologically safe addition. These single doses in sachets are packaged in airtight manner immediately after filling of the sachets for storage.

The in each case separately prepared, preferably formulated, active ingredient-containing dosage forms, which are multiparticulate but not present as active ingredient powder, may also preferably be introduced into the airtight packaged system according to the invention initially as single doses in the above-stated ratios by weight in a drinking straw. Correspondingly configured administration systems are described in the publications US 2005/109858 (=WO 03/079957), WO 2004/000202, and WO 2004/000264, the entire disclosures of which are incorporated herein by reference.

Preferably, the two separately prepared, preferably separately formulated, pharmaceutical, multiparticulate dosage forms, not present in powder form, are present particularly preferably in each case as a granular product or pellets preferably as a mixture in the drinking straw. Optionally, in such a combination the clavulanic acid-containing component may also be present solely as crystals of the active ingredient. To allow problem-free swallowing of such a mixture, the preferably multiparticulate dosage forms have a particle size of from 250 to 800 μm and are preferably spheronized to yield pellets. In this way, even patients with swallowing difficulties can take such a single dose completely in the necessary dosage by sucking the conveying liquid through the drinking straw.

Preferably, the drinking straw is provided with a retaining means for the pharmaceutical combination of dosage forms. This retaining means is preferably arranged movably in the drinking straw, such that by sucking up the conveying liquid it is easily possible to suck up not only the dosage forms, but also the retaining means, preferably in the form of a plug, preferably up to the mouth orifice, so making it readily possible to monitor complete intake of the dosage. Preferably, the retaining means is permeable to the conveying liquid and air but not to the dosage forms. The combination of dosage forms is arranged between the preferably mobile retaining means and the opening of the drinking straw, which serves as the mouth orifice. If the drinking straw comprises a bend, which is optionally reversible and has a concertina-like configuration, both the mobile retaining means and the dosage forms are arranged in the portion of the drinking straw which comprises the mouth orifice and is joined to the second portion of the drinking straw by means of the bend.

To ensure the storage stability of these embodiments of the system according to the invention, they also comprise, in addition to the drinking straw with the pharmaceutical combination, a preparation which includes the desiccant. Thus, the desiccant may preferably be admixed as a physiologically acceptable component, preferably as an edible component, optionally in multiparticulate form, with the combination of the two preferably granulated, particularly preferably pelletized, active ingredient-containing dosage forms as a further component, or be present in the drinking straw in a manner separated from the combination, preferably the mixture of the dosage forms, by the retaining means.

The desiccant may also be processed as a constituent of the drinking straw material, together with the preferably thermoplastic, physiologically safe polymers and other given constituents, by thermoforming to yield the drinking straw. The desiccant may also be used as a component of a drinking straw finish, such as for example as a constituent of the porous retaining means, preferably a porous plug, which consists substantially of non-woven material, filter material or pressed fibrous material, preferably synthetic fibrous materials.

Very particularly preferably, the desiccant is present in a further drinking straw finish, namely a closing device for the mouth orifice of the drinking straw. This closing device may take the form of a closing membrane or closing cap, which is removable, preferably a closing cap. Such a closing device, preferably a closing cap, of the drinking straw comprises the desiccant as one component of the composition thermoformed for this purpose. This composition comprises the desiccant, at least one water-insoluble, thermoplastic polymer and an agent incompatible with this polymer as a channel former, preferably an agent which is so much more hydrophilic relative to said polymer that the difference in hydrophilicity leads these two components to be so incompatible that they separate from one another.

The agent which leads to channel formation in the thermoplastic polymer may suitably comprise a compound from the group comprising polyglycol, ethylene/vinyl alcohol copolymer, glycerol, polyvinyl alcohol, pentaerythritol, polyvinyl pyrrolidone, a saccharinate, a polyhydric alcohol or an ethylene/vinyl acetate copolymer with preferably 4 to 40 mol % vinyl acetate units. The composition preferably comprises 1 to 10 wt. %, preferably 3 to 7 wt. %, particularly preferably 4 to 5 wt. % of the channel-forming agent. The use of an ethylene/vinyl acetate copolymer is preferred, since mechanical characteristics, such as flexibility, may also be positively influenced in this way.

The thermoplastic, preferably hydrophobic polymer, which is used to produce the closing device, preferably the closing cap, may suitable comprise a physiologically safe polymer, preferably a polyolefin, very particularly preferably a polyethylene or polypropylene. As a further component of the composition, from which the closing device, preferably the cap for the drinking straw, is formed, a desiccant, preferably amorphous silica, particularly preferably precipitated silica, such as silica gel, is present in such quantities that, on storage of the airtight packaged system under stress conditions of 40° C./75% relative atmospheric humidity for up to 3 months, at most 1.5 wt. % of the amoxicillin trihydrate is dimerized and at most 10 wt. % of the clavulanic acid is degraded.

This also applies to a necessary quantity of the desiccant, if present in the drinking straw as a component of the drinking straw or another drinking straw finish such as that for the retaining means. So that at least the unbound water of the aqueously formulated, amoxicillin-containing dosage form is absorbed by the desiccant, the water absorption capacity of the desiccant, irrespective of whether the latter is present as a component of the drinking straw or of the drinking straw finish, must amount to at least 25 mg water per gram of desiccant both at 40° C./75% relative atmospheric humidity and at 22° C./80% relative atmospheric humidity in each case over the first 24 hours of storage under the stated conditions and at least 40 mg water per gram of desiccant during subsequent storage for up to at a total of 7 days in each case under the stated conditions, the water absorption of the desiccant at most doubling over the first 24 hours or after a total of 7 days in each case on changeover of the storage conditions from 22° C./80% relative atmospheric humidity to 40° C./75% relative atmospheric humidity.

The melt-processable composition for producing the closing device, in particular the closing cap of the drinking straw, may comprise pigments, such as for example titanium dioxide and/or calcium carbonate in conventional pigment quantities, preferably up to 2 wt. %, relative to the total weight of the thermoformable composition, for coloring purposes.

Preferably, the chemical affinity of the desiccant for the channel-forming agent is greater than for the thermoplastic matrix polymer, such that, on melting of the thermoplastic polymer for thermoforming, during which the channel-forming agent also melts, the desiccant preferably aggregates with this agent and, after thermoforming, separates during cooling in the thermoplastic polymer, preferably the polyolefin, of which more than 50 wt. % of the thermoformable composition consists, and thus forms channels in the matrix polymer. The necessary absorption of moisture by the desiccant may thus take place without hindrance. The retaining means, which is preferably of plug-type construction, may also be produced in this manner.

However, it is also possible to add to the drinking straw filled with the pharmaceutical combination, prior to airtight packaging, a desiccant preparation of the stated desiccants with the stated water absorption capacity in the necessary quantities, preferably in the form of desiccant-containing strips, which also include suitable carriers.

However, the preferred embodiment according to the invention is an airtight packaged administration form in the form of a drinking straw with a closing device, preferably with a closing cap, provided with a desiccant. To improve handling and as a safety precaution in the case of inadvertent swallowing, this closing cap may also comprise lateral orifices, preferably in the form of slots.

### BRIEF DESCRIPTION OF THE DRAWING

FIG. 1 shows an oral administration form comprising a drinking straw.

### DETAILED DESCRIPTION OF AN ILLUSTRATIVE ADMINISTRATION FORM

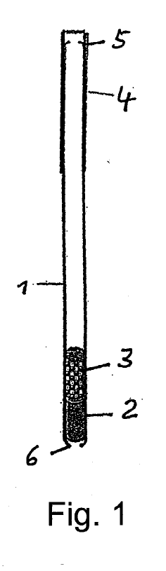

FIG. 1 depicts an oral administration form in the form of a drinking straw (1), in which is present a mixture of the pharmaceutical formulations containing on the one hand amoxicillin trihydrate and on the other hand potassium clavulanate in the form of pellets (3). This formulation is arranged above a mobile retaining means in the form of a plug (2). The mouth orifice, which the mixture of pharmaceutical preparations, preferably formulations, may reach without hindrance, is provided with a closing cap (4). This closing cap was made by thermoforming from a thermoplastic composition comprising a desiccant, as explained above.

Using the illustrated device, the mixture of pharmaceutical preparations, preferably formulations, may be taken in by the patient by sucking up a conveying liquid, which does not arrive in the drinking straw through the mouth orifice (5) but rather through the other opening (6) of the drinking straw, the mobile retaining means preferably moving at the same time as a “controller”. Before the conveying liquid is sucked up, it goes without saying that the closing device has to be removed from the mouth orifice of the drinking straw.

The desiccant-containing drinking straw finish may preferably be produced by injection molding, by mixing the thermoplastic matrix polymer, preferably a water-soluble polymer, preferably a polyolefin, such as polyethylene or polypropylene, preferably after it has previously been melted, with the further components, namely the channel-forming agent and the desiccant, preferably in multiparticulate form. To achieve a largely homogeneous distribution of the latter two components in the polymer melt, mixing takes place in known mixers with sufficient stirring. The molten mixture may be brought into the desired shape using an injection molding machine. The shaped articles, preferably the closing cap, for the drinking straw is cooled, wherein during cooling the channel-forming agent is separated and aggregated with the desiccant as a result of its greater affinity therefor. Channels are then formed in the thermoplastic matrix polymer, with which the agent is largely incompatible, said channels having an open connection with the surface of the injection-molded article and comprising an accumulation of desiccant. In this way, the desiccant may absorb the moisture from the surrounding atmosphere, such that a storage-stable, airtight packaged, oral administration form is achieved.

The retaining means arranged in the drinking straw, preferably a mobile plug, may also be produced in the same way. With regard to the production of such desiccant-containing moldings, containing channels, of thermoplastic polymers or corresponding compositions for producing such moldings, reference is made to publication WO 97/32663 or WO 99/61856. The description of the moldings or the methods for producing such moldings in the stated publications is hereby introduced as a reference and is deemed to be part of the present disclosure.

Suitable packaging materials, preferably multilayer films with a water vapor barrier layer, preferably of aluminum, for airtight packaging of the system according to the invention are known to a person skilled in the art.

### Method for Determining Water Content

Karl-Fischer water content is determined according to the publication in the European Pharmacopeia under no. 2.5.32 or. 2.5.12 depending on water content.

The active ingredient content and the quantity of the degradation products of potassium clavulanate is determined using HPLC.

The method for determining the dimer formation of amoxicillin trihydrate is based on HPLC.

To determine the storage stability of the system according to the invention, the active ingredient content of the two active ingredients is determined at a specific time using HPLC.

Active ingredient release is determined according to the European Pharmacopeia at a release medium temperature of 37° C. and a rotational speed for the paddle stirrer of the release apparatus of 100 min⁻¹ in the time stated in the Example and the release medium stated therein. The particular quantity of active ingredient released at a specific time was determined by HPLC.

### EXAMPLES

### Example 1

### A. Coated Amoxicillin Trihydrate Extruded Pellets

a) Extruded pellets having the following composition

| Per dose | Starting materials |
|---|---|
| 575.00 mg | Amoxicillin trihydrate Ph. Eur. (= 500 mg of |
| | amoxicillin, anhydrous) |
| 65.00 mg | Tricalcium phosphate, Ph. Eur. |
| 250.00 mg | Carrageenan NF, Ph. Eur. |
| | Purified water |

were produced by mixing the starting materials in a high speed mixer and then adding water to the mixture by wet granulation and extrusion of the moist granules through an extruder with a 0.5×0.5 mm extrusion die at extrudate temperatures of below 35° C. The moist extrudates were spheronized in a suitable spheronizer and the resultant pellets were dried at 105° C. in a fluidized bed down to a residual moisture content of below 10% using the IR method. The dried pellets were classified by the screening method and the 250 to 710 μm fraction of all the screening operations was combined.
b) The extruded pellets obtained according to a) were coated with a taste-masking coating comprising an aqueous dispersion having the following composition up to a weight gain of 2 wt. %, relative to the total weight of the pellets:

Composition of the Aqueous Coating Dispersions

| Per dose | | |
|---|---|---|
| 15.51 | mg as dry substance | Methacrylic acid/ethyl acrylate |
| | | copolymer 1:1, 30% aqueous dispersion, |
| | | (Eudragit ® L30 D-55), Ph. Eur. |
| 1.91 | mg | Triethyl citrate, Ph. Eur. |
| 0.36 | mg | Glycerol monostearate (Cutina V, |
| | | plant origin), Ph. Eur. |
| 0.02 | mg | Polysorbate 80 Ph. Eur. |

To this end, the pellets were provided with a coating by means of the aqueous coating dispersion with a 15 wt. % solids content in a fluidized bed installation with the introduction of warm air at a product temperature of 30° C. up to a weight gain of 2 wt. % and dried by the IR method with the introduction of a reduced amount of warm air until a product temperature of 40° C. and a residual moisture content of <10% are reached.

### B. Potassium Clavulanate Pellets

Pellets were produced with the following composition in a room with less than 20% atmospheric humidity and a room temperature of below 25° C.:

| Per dose | | Starting materials |
|---|---|---|
| 12.7 | mg | sucrose stearate (di-, tri-, |
| | | polyester/HLB 1) |
| | | melting range 51 to 61° C. |
| | | (sucrose ester S170) |
| 74.45 | mg (\hat{=} 62.5 mg clavulanic acid) | potassium clavulanate |
| 12.7 | mg | Kaolin |

Pellets with the foregoing composition were produced by preheating a 4 liter Diosna mixer to 60° C. and then mixing sucrose ester and kaolin at a mixing power of 650 revolutions per minute and a chopper speed of 1000 revolutions per minute until the mixture was melted. The mixture was cooled to room temperature (25° C.) and added to the mixture heated to approx. 60° C. of K clavulanate and granulated and rounded at <65° C., a mixing power of 500-700 revolutions per minute and a chopper speed of 1000 revolutions per minute. The finished pellets were rapidly cooled within <3 min to 30° C. by introduction of liquid N₂ while occasionally being mixed without turning on the chopper.

The resulting pellets displayed a narrow size distribution after classification using the screening method. All the screening fractions from 250 to 800 μm were stored in airtight containers, which were provided with desiccant.

### C. Production of the Airtight Packaged System

a) Amoxicillin trihydrate-containing pellets, produced as set forth in A, and potassium clavulanate-containing pellets, produced as set forth in B, are introduced into a transparent drinking straw, which has a mobile, porous controller inside it, at temperatures of below 20° C. and at a relative atmospheric humidity of ≦20%, such that the ratio of amoxicillin to clavulanic acid amounts to 8:1 and the absolute dose amounts to 500 mg amoxicillin and 62.5 mg clavulanic acid as a single dose. The orifice which the mixture of the pellets may reach without hindrance (=mouth orifice) is provided with a closing cap immediately after filling.
b) This cap was made from the following composition using an injection molding machine and a corresponding cap mold:

| Per cap | |
|---|---|
| 412.5 mg | silica gel (Syloids ®) |
| 610.5 mg | polypropylene |
| 8.25 mg | calcium carbonate |
| 10.00 mg | titanium dioxide |
| 55.00 mg | ethylene/vinyl acetate copolymer |
| | with 28 mol % vinyl acetate units |

The components were mixed with heating until the polymers were melted and the melt could be conveyed effortlessly to the corresponding molds for shaping. During cooling of the caps in the mold, the incompatibility of the polypropylene with the ethylene/vinyl acetate copolymer resulted in the formation of channels, in which the desiccant accumulated because of its greater affinity for the ethylene/vinyl acetate copolymer than for the polypropylene. The caps were stored at a relative atmospheric humidity ≦ of 20% or sealed hermetically in the aluminium pouch.

The drinking straw filled as set forth in C is closed with the desiccant-containing cap and packaged in airtight manner by sealing in a packaging material of a multilayer film, which includes at least one layer as a water vapor barrier layer (=an aluminium layer with a thickness of 20 μm).

### D. The Airtight Packaged Systems According to the Invention Were Subjected to a Plurality of Tests.

a) The water absorption capacity of the closing cap, which is used to close the airtight packaged system according to the invention, was determined at 40° C. and 75% relative atmospheric humidity or at 22° C. and 80% relative atmospheric humidity using the above-stated Karl Fischer method after storage of 1 day or up to a total of 7 days. The corresponding values may be found in Table 1.

TABLE 1
Water absorption of a 1.1 g cap

| After 24 h | After 7 days | After 24 h | After 7 days |
|---|---|---|---|
| 22° C./80% RH | 22° C./80% RH | 40° C./75% RH | 40° C./75% |
| | | | RH |
| 42 mg | 62 mg | 58 mg | 96 mg |

b) Moreover, the airtight packaged systems according to the invention were stored for up to 3 months at 40° C. and 75% relative atmospheric humidity and at the times stated in Table 2 in each case the content of amoxicillin trihydrate was determined by HPLC and the dimers formed as degradation products as a result of dehydration were determined by HPLC. The content of clavulanic acid and the degradation products of the clavulanic acid arising during decomposition were determined by HPLC and stated in Table 2.

TABLE 2

| | | |
|---|---|---|
| 0 | 1 month | 3 months |

Example 1 Storage at 40° C./75% RH after

| | | | |
|---|---|---|---|
| Amoxicillin | 98.9% | 99.3% | 96.9% |
| Clavulanic acid | 102.8% | 103.7% | 101.1% |
| Dimers of | — | 0.37% | 0.30% |
| amoxicillin | | | |
| Degradation | 0.48% | 0.66% | 0.63% |
| products of | | | |
| clavulanic acid | | | |

Comparative Example 1 Storage at 40° C./75% RH after

| | | | |
|---|---|---|---|
| Amoxicillin | 100.9% | 100.7% | 100.7% |
| Clavulanic acid | 99.0% | 90.0% | 63.3% |
| Dimers of | 0.25% | 0.17% | 0.13% |
| amoxicillin | | | |
| Degradation | 0.32% | 0.65% | 0.98% |
| products of | | | |
| clavulanic acid | | | |

### Comparative Example 1

In comparison thereto, a system identical to the system according to the invention was produced, with the exception of the closing cap, which was made from the composition mentioned in C without desiccant. The airtight packaged system was likewise stored at 40° C. and 75% relative atmospheric humidity for up to 3 months and the corresponding values entered in Table 2.

### Example 2

As indicated in Comparative Example 1, a corresponding administration form was provided in a drinking straw. The closing cap of the drinking straw does not contain any desiccant.

Prior to packaging of the drinking straw in a packaging material, as described in Example 1 D, at temperatures of below 25° C. and a relative atmospheric humidity of 15%, an 8 cm long, 33 mm wide and 0.3 mm high (volume 792 mm³) silica gel strip was added to the packaging immediately prior to packaging of the drinking straw, the water absorption capacity of which strip after 7 days storage at 22° C./80% relative atmospheric humidity was 129 mg. The sealed packages were subjected to the Tests as stated in Example 1. The corresponding values are stated in Table 3.

TABLE 3
Storage at 40° C./75% RH after

| | 0 | 1 month | 3 months |
|---|---|---|---|
| Example 2 | | | |
| Amoxicillin | 99.6 | 100.7% | 99.6% |
| Clavulanic acid | 96.9% | 95.9% | 93.3% |
| Amoxicillin dimers | 0.37% | 0.34% | 0.27% |
| Degradation products | 0.54% | 0.3% | 0.59% |
| of clavulanic acid | | | |
| Comparative | | | |
| Example 2 | | | |
| Amoxicillin | 107.2% | 101.4% | |
| Clavulanic acid | 99.0% | 97.1% | |
| Dimers of amoxicillin | 0.19% | 1.83% | |
| Degradation products | 0.22% | 0.45% | |
| of clavulanic acid | | | |

### Comparative Example 2

As stated in Example 2, an administration form was produced and packaged by sealing in a packaging containing a corresponding desiccant trip 8 cm in length, 24.5 mm wide and 0.4 mm high (volume 784 mm³), which strip contained as desiccant a molecular sieve (Grade 4 Å, Siliporit NC 10®) made by Ceca Arkema Groups). The storage stability values measured using the above-stated method are listed in Table 3.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. An airtight packaged system comprising:
(1) a combination of the two active ingredients, amoxicillin trihydrate and potassium clavulanate, which are not present as a powder mixture, but as two in each case mutually separately prepared pharmaceutical dosage forms, which are multiparticulate but not present as active ingredient powders and which in each case consist of an aqueously formulated oral dosage form containing amoxicillin trihydrate as a pharmaceutical active ingredient and of a dosage form prepared, preferably formulated, separately therefrom and containing as pharmaceutical active ingredient potassium clavulanate used undiluted with colloidal silicon dioxide or microcrystalline cellulose as desiccant, the active ingredients being present in the combination in a nominal weight ratio of amoxicillin to clavulanic acid of from 20:1 to 1:1, and
(2) a preparation comprising at least one desiccant in such quantities that, during storage of the packaged system under stress conditions of 40° C./75% relative atmospheric humidity for up to 3 months, amoxicillin trihydrate dimerises at most up to 1.5 wt. % and the clavulanic acid is degraded to at most 10 wt. %, the water absorption capacity of the desiccant amounting to at least 25 mg water/g desiccant both at 40° C. and 75% relative atmospheric humidity and at 22° C. and 80% relative atmospheric humidity in each case over the first 24 hours of storage under the stated conditions and at least 40 mg water/g desiccant during subsequent storage for up to at total of 7 days in each case under the stated conditions, the water absorption capacity of the desiccant in each case at most doubling when the storage conditions are in each case changed from 22° C./75% relative atmospheric humidity to 40° C./75% relative atmospheric humidity over an identical period.

2. An airtight packaged system according to claim 1, wherein the amoxicillin and clavulanic acid are present in a nominal weight ratio of from 12:1 to 2:1.

3. An airtight packaged system according to claim 1, wherein the amoxicillin and clavulanic acid are present in a nominal weight ratio of 8:1, 7:1, 6:1, 5:1, 4:1, 3:1 or 2:1.

4. An airtight packaged system according to claim 1, wherein the desiccant is present at least in such quantities that, during storage at 40° C. and 75% relative atmospheric humidity, the content of unbound water of the combination is absorbed from the pharmaceutical dosage forms.

5. An airtight packaged system according to claim 1, wherein the desiccant is based on an amorphous silica.

6. An airtight packaged system according to claim 5, wherein the amorphous silica is a precipitated silica, a silica gel, an amorphous, pyrogenic silica or a mixture of two or more thereof.

7. An airtight packaged system according to claim 1, wherein each of the two dosage forms is present in a quantity corresponding to a single dose.

8. An airtight packaged system according to claim 1, wherein the clavulanic acid-containing dosage form comprises crystals of clavulanic acid or a pharmaceutically acceptable salt thereof.

9. An airtight packaged system according to claim 1, wherein each of the two multiparticulate dosage forms is present in the form of granules or pellets.

10. An airtight packaged system according to claim 1, wherein the combination of the dosage forms is readily dispersible or at least partially soluble in an aqueous liquid.

11. An airtight packaged system according to claim 1, wherein the combination of the dosage forms may be readily conveyed with aqueous liquid.

12. An airtight packaged system according to claim 1, wherein said system comprises a drinking straw, in which the combination of dosage forms is arranged.

13. An airtight packaged system according to claim 12, wherein the combination of dosage forms is in the form of granules or pellets or a combination of granules or pellets containing amoxicillin trihydrate and clavulanic acid-containing crystals.

14. An airtight packaged system according to claim 12, wherein the drinking straw is provided with an optionally movable retainer for the combination of the dosage forms.

15. An airtight packaged system according to claim 14, wherein said retainer is a plug-like controller.

16. An airtight packaged system according to claim 12, wherein at least one opening of the drinking straw is provided with a removable closure.

17. An airtight packaged system according to claim 16, wherein said closure comprises a membrane or cap optionally comprising a weakened portion for facilitating removal of the membrane or cap.

18. An airtight packaged system according to claim 12, wherein at least part of the desiccant is arranged in the drinking straw, or is present as a constituent of the drinking straw or of a finishing element of the drinking straw.

19. An airtight packaged system according to claim 14, wherein the desiccant is present in the drinking straw and is separated by the retainer from the combination of the two dosage forms.

20. An airtight packaged system according to claim 14, wherein the desiccant is a further component of said mixture, or is a component of the retainer, or a component of a closure for the drinking straw.

21. An airtight packaged system according to claim 20, wherein the desiccant is present as a component of a closing cap, which closes the mouth orifice of the drinking straw which offers unimpeded access to the combination of dosage forms.

22. An airtight packaged system according to claim 16, wherein the closure is molded from a composition comprising the desiccant, at least one water-insoluble, hydrophobic, thermoplastic polymer and an agent incompatible therewith as channel former for the thermoplastic polymer.

23. An airtight packaged system according to claim 22, wherein said composition is molded by thermoforming to produce the closure in the form of a cap for the drinking straw.

24. An airtight packaged system according to claim 22, wherein the channel-forming agent is so incompatible with the thermoplastic polymer that the channel forming agent and the thermoplastic polymer separate from one another.

25. An airtight packaged system according to claim 22, wherein the channel forming agent comprises a polyglycol, an ethylene/vinyl alcohol copolymer, glycerol, polyvinyl alcohol, pentaerythritol, polyvinylpyrrolidone, a saccharinate or a polyhydric alcohol.

26. An airtight packaged system according to claim 22, wherein the closure for the drinking straw comprises from 1 to 10 wt. % of the channel-forming agent.

27. An airtight packaged system according to claim 26, wherein the closure for the drinking straw comprises 4 to 5 wt. % of the channel-forming agent.

28. An airtight packaged system according to claim 26, wherein the closure of the drinking straw contains the desiccant in such a quantity that, during storage of the airtight packaged system under stress conditions of 40° C. and 75% relative atmospheric humidity for up to 3 months, at most 1.5 wt. % of the amoxicillin trihydrate dimerises and at most 10 wt. % of the clavulanic acid is degraded.

29. An airtight packaged system according to claim 18, wherein desiccant in the drinking straw has a water absorption capacity of at least 25 mg water per gram of desiccant at 40° C. and 75% relative atmospheric humidity or at 22° C. and 80% relative atmospheric humidity within the first 24 hours of storage under the stated conditions and at least 40 mg water per gram of desiccant during subsequent storage up to a total of 7 days under the stated conditions.

30. An airtight packaged system according to claim 1, wherein the airtight package comprises a packaging material with a water vapor barrier layer of aluminum.
